(19) United States
(12) Patent Application Publication
(10) Pub. No.: US 2014/0030347 A1
(43) Pub. Date: Jan. 30, 2014

(54) MASS PRODUCTION OF READY-TO-USE SUSPENSIONS OF FIBRINOGEN-COATED ALBUMIN SPHERES FOR THE TREATMENT OF THROMBOCYTOPENIC PATIENTS

(71) Applicant: Richard C.K. Yen, Yorba Linda, CA (US)

(72) Inventor: Richard C.K. Yen, Yorba Linda, CA (US)

(21) Appl. No.: 13/560,727

(22) Filed: Jul. 27, 2012

(51) Int. Cl. A61K 38/38; A61P 7/02; A61K 9/14

(52) U.S. Cl. 424/491; 514/13.6

(57) ABSTRACT

A composition and a method effective in the production of the composition. The composition is a ready-to-use aqueous suspension in large and small quantities comprising human-fibrinogen-coated human-albumin spheres and the supernatant, said suspension being useful for the treatment of thrombocytopenic patients.

### BACKGROUND OF THE INVENTION

1. Field of the Invention

The field of this invention concerns suspensions of fibrinogen-coated albumin spheres and a suitable supernatant useful in the therapeutic treatment of thrombocytopenic patients and the prophylactic treatment of patients who are expected to become thrombocytopenic due to a variety of reasons. The cause of thrombocytopenia may be external bleeding, such as from trauma, war situations, or during surgery. It may be due to internal conditions, such as cancer, cancer treatment, damage to the bone marrow after exposure to high doses of radiation, sepsis, disseminated intravascular coagulation, burn, infection from virus (e.g. HIV, Ebola, Dengue), chemical agents or environmental causes. Treatments with fibrinogen-coated albumin spheres in some of these patients have already been shown to improve the patients' condition, such as decreased bleeding time, reduced bleeding volume, faster recover, and improved survival rate.

Specifically, this invention involves the invention of a new suspension formulation which comprises a controlled reduction in the size of the spheres as well as the composition of the excipient components in the fluid phase of the suspension. This new invention is different from all prior art disclosures where the product has been a lyophilized powder which requires reconstitution with a fluid to become a suspension before the prior-art product in the suspension format can be administered to a patient. In addition, the reconstituted suspension must be used within a defined time limit before some large spheres in the suspension begin to settle and form sediments at the bottom of the container. In contrast, the invention in this patent application is a ready-to-use formulation which does not require the step of reconstitution. The spheres in the suspension also do not form settled particles at the bottom of the container despite prolonged storage in room temperature without shaking. Both the spheres and all the excipient components in the suspension can be subject to a heat treatment. The heat treatment is effective in the inactivation of any potential infectious agents that get into the suspension any time before the suspension is sealed inside a container.

Specifically, this invention also discloses the method of production of massive quantities of the suspension with great consistency and reproducibility. The resulting product does not contain more than 0.1% of its spheres having a diameter larger than one micron and does not contain any aggregates.

Specifically this invention also comprises a step of terminal sterilization which cannot be performed on the products disclosed in all prior arts which are lyophilized products.

This invention has many advantages, which include: (a) the product can be administered to a patient without the need to reconstitute a dry powder with a defined volume of fluid; (b) this product, as a ready-to-use formulation, can be administered to a large number of patients within a short time and without the delay such as when the other bottle containing the reconstitution fluid is broken or lost; (c) no need to involve specially-trained personnel to learn how exactly to perform the process of reconstitution; and (d) the product will not be wasted if not used; in contrast to a reconstituted suspension which will be wasted if not used within a defined time because the reconstituted product contains large spheres which can settle after reconstitution in a suspension. In addition, this invention permits the product to be (e) stored for over one year in room temperature without detectable loss of activity; (f) the step of “terminal sterilization” will add to the safety of the product from infectious agents. Moreover, the removal of the step used in the prior art, which is the step of lyophilization, will (g) decrease the cost of production and transportation which will translate directly to a reduction in the cost associated with treatment and the care of thrombocytopenic patients in the national health system.

This invention is novel and non-obvious because it involves a product and a method of production of the product which is a suspension containing spheres and a supernatant fraction both of which are compatible for use, even after heat treatment, directly to patients. The invention discloses teaching that are directly opposite to what was taught in the prior art and the size of the spheres in the invention is smaller than that of natural platelets and works even better than donor platelet transfusions.

2. Description of the Prior Art

Numerous inventors have attempted to produce albumin spheres suitable for intravenous administration to patients. Problems encountered with these early products made them unsuitable for most medical uses, far less for use as artificial platelets. However, prior art spheres were prepared by emulsification of protein solutions in oil, followed by heating or extensive polymerization to harden the protein droplets. As such, they appear to the body as foreign particles and will be removed from the circulation within minutes after intravenous administration. Therefore, there is a need for better methods of production leading to particles that are “benign” to the defense system of the body and which allows the particles to stay for prolonged periods in the blood stream without destruction or removal.

Zee patents (“A Novel Device for Promoting Healing From Surgical and Medical Treatment, Its Use and Method of Production”, applied for in China 1995; “A Method for Promoting Healing From Surgical and Medical Treatment, Its Use and Method of Production”, applied for in China 1995) describe a specific type of albumin spheres suitable for use as artificial platelets. However, the method of production includes the addition of a surfactant, e.g. sodium tetradecyl sulphate or Tween-80 to ensure that the spheres do not form aggregates greater than 7 micron in diameter which can clog blood vessels. Zee also disclosed that the product made by his method requires lyophilization for long term storage. Although Zee claimed that the “average” size of the product made by his method is less than one micron, it is obvious that the product contains some large spheres which are larger than one micron. Spheres larger than one micron cannot remain suspended for long by the Brownian movement of water molecules. Regardless of the percentage occupied by these large spheres, these larger-than-one micron spheres will quickly settle to the bottom of the container. It is not possible to separate such sediments back into single spheres by merely shaking the container. The presence of clumps in the settled layers can cause obstruction of blood vessels if administered intravenously to a patient. As a result, the product of Zee must be lyophilized soon after their synthesis during the entire manufacturing process and the dried powder needs to be converted back into a suspension by reconstitution with a fluid at a time immediately before administration to a patient. The spheres within a reconstituted suspension manufactured by Zee will sediment into a bottom layer which is visible by the unaided eye within 8 hours. The rubber cap used to seal the content of the bottle will be punctured twice: once for the introduction of the fluid for reconstitution, a second time for the withdrawal of the reconstituted suspension. Therefore, there is an increased chance of contamination, particularly during the time between the first puncture and the second puncture, where the cap with the first puncture hole is exposed and unprotected. Therefore, there is a need for improved products such as from this invention which is comprised of (a) spheres which do not settle to form sediments for prolonged periods, (b) both spheres and the supernatant can be subjected to a terminal sterilization without damage, and (c) the cap of the container and all “barriers” will not be broken until the patient is ready to be administered the product.

The need to lyophilize a product has many disadvantages. (1) This step consumes a large amount of electricity. (2) During the few days needed to “pull” water molecules from the frozen preparation, the content inside the bottle is exposed because the cap of the bottle has to sit loosely on top of the bottle in order for the water molecules to escape from the bottle. Any airborne particles inside the lyophilizer can potentially get inside a bottle before the cap of the bottle is pressed firmly into the bottle to seal the content of the bottle at the end of the lyophilization step. (3) An extra bottle of sterile fluid must be provided so that the health provider can reconstitute the dry powder back to a suspension; this increases the cost of production and the cost of transportation. (4) During stressful conditions, such as during battle or after a massive natural disaster, the extra bottle of fluid may be lost, stolen or broken, making it impossible to use the dry product. (5) It takes time for the dry power to be properly reconstituted. If there is a large number of patients, the health provider may be tempted to start to administer a partially reconstituted powder intravenously to a patient, which will not only reduce the effective number of fibrinogen-coated spheres available to perform their health benefits but it will cause obstruction of blood vessels in the patient. (6) Any reconstituted product must be used within a limited time or else the larger particles (larger than one micron in diameter) will settle. (7) Reconstituted products that are not used will have to be wasted because the sterility barrier has been broken by the step of reconstitution. In addition, after a suspension has been lyophilized and the bottle containing the dried powder is capped, terminal sterilization cannot be performed—there no effective way of killing germs mixed with a dry powder such as a lyophilized powder within a sealed bottle.

Therefore, there is a need for a new product which (a) avoids the use of surfactants which some patients may be allergic to; (b) comprises spheres of even smaller dimensions (with less than one percent of the sphere population being larger than one micron in diameter) that can stay in suspension over a long period; (c) allows a step of terminal sterilization which will kill any infectious agents that might have entered the bottle during the filling of the bottle with the suspension product.

However, producing spheres nearly all of which (or all of which) are less than one micron will be technologically challenging. Most particles in nature are either larger than one micron or less than 10 nanometer in size. This is due to the physical forces governing the formation of stable particles on earth. For example, bacteria are typically larger than one micron. Even the platelets inside the patient's body are typically two micron in diameter. There is no easy way to produce stable particles in the range between 0.01 micron and one micron in diameter. In addition, there is no guarantee that a particle substantially smaller than the natural platelet can be effective in reducing bleeding time in thrombocytopenic patients. This is because the mass of a particle decreases substantially when the size decreases, while its “surface area per mass” ratio increases dramatically. A particle that is one micron in diameter (with a density of one) will have a mass of 5.2 E-10 milligram and a surface area of 3.1 E-6 millimeter square (with a Surface Per Mass ratio of 0.6 units.) By comparison, a particle that is one quarter as big (i.e. 0.25 micron in diameter) will have a mass of only 0.08 E-10 milligram and a surface area of 0.2 E-6 millimeter square, producing a Surface Per Mass ratio of 2.5 units. However, if the technology can be developed to produce small spheres consistently, as in the present invention, it will result in a product that can be highly effective: because for the administration of every milligram of product, a product with an average diameter of 0.25 micron will have many more particles administered than a product with an average diameter of one micron. The smaller-size product will also have a much larger “total” surface area (i.e. the sum of all the surface areas of all the smaller spheres administered.) The smaller-size product will be particularly effective in applications where the medical benefit is related to the availability of surfaces for binding with other biological material, such as with the surface of activated platelets at a wound site. Spheres smaller than one micron also have the advantage that they will circulate closer to the endothelium of the blood vessel than even natural platelets, which are typically two micron in diameter. This is because particles flowing in a “tube” will tend to sort themselves out according to rheological principles where larger particles (such as red cells) will flow near the center of the tube (such as a blood vessel) and smaller particles (such as platelets) will flow near the wall of the tube. This is important because wounds occur on the wall of the blood vessels and not at the center of blood vessels. Therefore, the product of this invention will be able to plug a wound on the blood vessel even more efficiently than natural platelets.

Inventor Yen has disclosed numerous methods of making albumin spheres, all of which included the use of detergents added to the protein solution to prevent the formation of aggregates when the desolvation agent (alcohol solution) is added to the protein solution. One such example is “Fibrinogen-coated Microspheres” U.S. Pat. No. 6,264,988B1. The one exception where surfactants were not added to the protein solution before the desolvation agent is added to the protein solution is U.S. Pat. No. 6,391,343B1 “Fibrinogen-coated Particles For Therapeutic Use.” In the only exception where surfactants were not used, Yen used a number of chemicals and drugs as stabilizing agents. Such agents include reducing agents, oxidizing agents, phosphorylated compounds, sulfur containing compounds, polymers and combinations thereof (see column 4, line 6-11.) The biological effects of these agents (or their residuals) when given at the same time with the sphere preparation have not been evaluated. Some of the agents may be toxic when administered in such a situation to patients. The major idea disclosed in U.S. patent 343B1 is that agents other than a known cross-linking agent (e.g. glutaraldehyde) can be used to stabilized spheres against resolubilization when the concentration of alcohol is reduced, and against the formation of aggregates during the formation of the spheres from soluble protein molecules (Column 4, line 41-49.)

Therefore, any process involving the production of spheres where the spheres remain in single-particle condition without the formation of aggregates, and where the spheres are stabilized by glutaraldehyde (and not the list of agents listed in patent 343B1) without the addition of surfactants is novel and non-obvious according to the teachings of all prior arts. In other words, if a prior art insists on the inclusion of a step for its success, a new process that is successful without this critical step must be regarded as a novel, non-obvious, and patentable process.

Yen has also disclosed a method of making large quantities of spheres (see U.S. Pat. No. 5,716,643, “Large Scale Production of Medicine Coated Crosslinked Protein Microspheres” and U.S. Pat. No. 6,013,285, “Large Scale Production Process with Instantaneous Component Mixing & Controlled Sequential Mixing Characteristics”.) However these prior arts only taught production methods using a tubing system. Tubing systems can be cumbersome particularly when the time between the addition of one component and the next component is long—the practitioner has to either reduce the pump rate or increase the diameter of the tubes so that the amount of tubing is not overbearing. However, larger diameter tubing allows mixing within the lumen of the tubing and will cause inaccuracy in the timing of the addition of the next and the following components. Therefore, there is need for a more simple system which guarantees thorough mixing of large quantities of liquid components, which can be achieved even though the length of time in between the addition of various components are very long (or very short.)

Many inventors have also disclosed methods of inactivating viral and bacterial agents by the use of heat or high pressure. These include the list (line 0004 to 0010) provided by Yen in “inactivation of Infectious Agents in Plasma Proteins by Extreme Pressure” (Publication number: US 2011/0251127 A1). However, none have taught how to inactivate infectious agents that may be hiding inside a sphere or have bound to the surface of a sphere (using the surface of a sphere as a protective layer.) In fact, Yen disclosed only a method to inactivate infectious agents associated with spheres only by extreme high pressure, which will not work with conventional glass containers. Glass containers will break under high pressure. Therefore, there is a need to discover improved methods of inactivating potential infectious agents introduced during the step of filling a glass container with a suspension. In addition, the process used to inactivate the infectious agent must not lead to the production of new antigen on the surface of the sphere or any components in the excipient molecules. Otherwise the product as a whole will not be suitable for use in a patient for single (one-time) administration or for repeated use in the same patient.

### SUMMARY OF THE INVENTION

The present invention is a composition and a method effective in the production of the composition. The composition is a ready-to-use aqueous suspension in large and small quantities comprising human-fibrinogen-coated human-albumin spheres and the supernatant, said suspension being useful for the treatment of thrombocytopenic patients. The cause of thrombocytopenia can be due to a variety of external or internal factors.

The size of the sphere population produced (when the quantity of production is less than ten liters in volume) has a typical range of less than one micron to less than 0.1 micron in diameter. Long term storage of more than one year in room temperature does not result in a visible bottom layer due to the sedimentation of any the spheres.

The size of the sphere population produced (when the quantity of production exceeds ten liter in volume) has a range of typically about one micron to less than 0.1 micron in diameter, with less than one tenth of one percent of the population being larger than one micron. Although a small minority of spheres may have a size larger than one micron, these larger spheres in the suspension remain in suspension due to the high concentration of spheres smaller than one micron and the suspension does not need to be stirred or agitated during at least six months of storage in room temperature.

In addition, the spheres in the composition have a surface that has never been exposed directly to air, having never been dried (including being lyophilized) and having never been prevented from or kept away from the direct contact with water. In other words, the spheres have always been in direct contact with an aqueous medium since the formation of the spheres, reducing any chance of conformational changes in the spheres which may induce immunological responses after administration to a patient.

Furthermore, the composition can be subject to a heat treatment sufficient to kill infectious agents associated with the spheres without causing surface changes that can lead to antibody formation in a human body when subsequently administered to a human body.

It has been found according to the present invention that a method of adding desolvation agent in two steps is superior to the prior art where the desolvation agent was added in one single step to the protein solution; the two-step method allows a much larger amount of desolvation agents to be used, producing a higher yield of spheres (above 80% and up to almost 100%) while at the same time producing smaller spheres (all of which are less than one micron in diameter) and no aggregates.

It has been found according to the present invention that the composition contains spheres of such small sizes that no sediments of spheres are observed at the bottom of the container after a prolonged period of storage such as for at least six months, during which time the containers are not deliberately shaken or agitated to shake up the spheres inside the container.

It has been found according to the present invention that human fibrinogen molecules added as a solution to the suspension of albumin spheres as late as one hour (or later) after the formation of the albumin spheres can still bind spontaneously, immediately and irreversibly to the albumin spheres without causing any loss in the yield of the albumin spheres in the suspension.

It has been found according to the present invention that a suspension of fibrinogen-coated albumin spheres can be mass-produced without the formation of aggregates when the volume of the component liquids are over 10 liters each and when the component liquids are added to each other in the correct sequence and with the correct timing, resulting in a final volume of at least 100 liters of the useful product.

It has been found that a ready-to-use suspension of fibrinogen-coated albumin spheres produced by the above mentioned, mass-production method can be filled to a glass container and sealed, followed by treatment of the entire sealed assembly with heat up to 65 degrees for 12 hours without suffering any loss of medicinal activity of said fibrinogen-coated albumin spheres.

It has been found according to the present invention that the heat treatment described above can result in the inactivation of said infectious agents hiding inside the interior of a sphere without causing a reduction in the medicinal properties of the spheres and without producing antigenicity in the spheres.

It has been found according to the present invention that the heat treatment described above can also result in the inactivation of said infectious agents associated with the surface of a sphere without causing a reduction in the medicinal properties of the spheres and without producing antigenicity in the spheres.

It has been found according to the present invention that the intravenous administration of the present invention at a dose of 4 mg per kg weight of the patient, or a higher dose, will result in improvements in the condition of patients who need platelet transfusions. Specifically the size of the spheres being less than one micron will allow the spheres to circulate nearer or closer to the wall of the blood vessels than even natural platelets, thus being more effective in forming plugs against any leaks or wounds in the wall of the blood vessels anytime and anywhere such leaks or wounds may occur.

Further novel features and other objects of the present invention will become apparent from the following detailed description, discussion and the appended claims.

### DETAILED DESCRIPTION OF THE PREFERRED EMBODIMENTS

Although specific embodiments of the present invention will now be described, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

### Experiment One

### Production of Spheres in a Suspension all of which are Smaller than One Micron in Diameter by the Addition of Desovation Agent in Two Steps

Purpose: To evaluate whether adding all the desolvation agent in one step is similar to, or worse than adding the desolvation agent in multiple steps (divided portions).

Materials and Methods: Human Serum Albumin (HSA) was purchased from commercial vendors including Alpha Therapeutic Corp, Los Angeles; Baxter Healthcare Corp, Glendale; Central Lab, Blood Transfusion Services, Swiss Red Cross; Immuno-US, Inc., Rochester; ZLB Bioplasma, Switzerland. Glutaraldehyde (GL) was purchased from Electron Microscopy Sciences, Fort Washington, Pa and Sigma-Aldrich, St. Louis. Protein concentrations were measured using the BCA method from Pierce Company. To obtain the yield of the reaction, spheres were removed by high speed centrifugation to obtain the clear supernatant fraction. The difference between the total concentration of protein per ml (spheres plus supernatant) and that of the supernatant fraction was the concentration of spheres per ml. The yield of the reaction is the concentration of the spheres (in milligrams per ml) divided by the concentration of the total protein (in milligrams per ml), expressed as a percentage.

An aliquot of 25% HSA was removed from a bottle purchased from each of the above commercial vendors and diluted with distilled water to 9%. GL purchased from the above vendors was also diluted with distilled water to 0.05%. The experiment was =lied out in room temperature varying from 18 degrees to 23 degrees Centigrade. Aliquots of 100 microliters of the 9% HSA were placed in small Eppendorf tubes. At time zero, 100 microliters of GL (0.05%) was added to the tube and mixed thoroughly with the HSA solution.

Experiment 1A: Addition of the desolvation agent in one step. At time equal to 60 seconds after the addition of GL, various volumes of Ethanol (70% in water) were added to the mixture according to Table One.

TABLE ONE
Volume of ethanol solution added to the HAS + GL mixture in one step

| | Volume of | Final | |
|---|---|---|---|
| | Ethanol Solution | Concentration of | |
| Tube number | (microliter) | Ethanol | Suspension |
| 1 | 300 | 42.0 | Mildly turbid |
| 2 | 325 | 43.3 | Spheres formed |
| 3 | 350 | 44.5 | Aggregates |
| | | | Observed |
| 4 | 375 | 45.7 | Massive |
| | | | Aggregates |
| 5 | 400 | 46.7 | Massive |
| | | | Aggregates |

Experiment 1B: Addition of the desolvation agent in divided portions (two steps). At time equal to 60 seconds after the addition of GL, the first portion of Ethanol (70% in water)—a non-precipitating amount which produces no turbidity in the mixture, was added to the mixture, mixed well, and then followed by the addition of a second portion of Ethanol (70% in water) at time equal to 150 seconds after the addition of GL, as listed in Table Two.

TABLE TWO
volume of ethanol used to prepare spheres in divided-portions approach

| | Volume of | Volume of | | Concentration | | |
|---|---|---|---|---|---|---|
| | ethanol, | ethanol, | Total vol of | of | | |
| | first | second | ethanol | Ethanol in | | |
| | portion | portion | added | final | | |
| Tube | (microliter) | (microliter) | (microliter) | solution, % | Suspension | Aggregate |
| 10 | 50 | 0 | 250 | 38.9 | Clear, not | None |
| | | | | | turbid | |
| 11 | 250 | 50 | 300 | 42.0 | Mildly | None |
| | | | | | turbid | |
| 12 | 250 | 100 | 350 | 44.5 | Turbid | None |
| 13 | 250 | 150 | 400 | 46.7 | Turbid | None |
| 14 | 250 | 200 | 450 | 48.5 | Turbid | None |
| 15 | 250 | 250 | 500 | 50.0 | Turbid | None |
| 16 | 250 | 300 | 550 | 51.3 | Turbid | None |
| 17 | 250 | 350 | 600 | 52.5 | Turbid | None |

Results:

Microscopic examination of the suspensions revealed that there were no spheres in tube 10 and very few spheres in tube 1 and 11. The spheres in tube 2 was about 2 micron in diameter, while those in tube 3, 4, 5 were larger than 2 micron with large numbers of aggregates of spheres which will render the suspension unsuitable for intravenous administration to a patient because of the presence of aggregates larger than 7 micron in diameter.

In contrast, the spheres in tube 12, 13, 14, 15, 16, 17 were all smaller than one micron and had no detectable aggregates present.

The yield of the experiment (mg spheres per ml divided by mg total protein per ml) in tube 2 obtained by the one-step approach was 31%, while that of tube 12, 13, 14, 15, 16, 17 obtained by the divided-portion method were measured to be 70%, 76%, 85%, 92%, 95% and 98%, respectively.

Comments:

While the results of using HSA supplied by different vendors show variation in the size of the spheres formed as well as in the yield, the results in this experiment apply to HSA supplied by all vendors: the superiority of the divided-portion method over the one-step method is clear. In the one-step method, aggregates were formed when the final concentration of ethanol reached 44.5% (as in tube 3). In contrast, no aggregates were seen in the divided-portion approach when the final concentration of ethanol exceeds 44.5% (as in tube 12) and even at 52.5% (as in tube 17.) The yield of the spheres in any reaction appears to be directly related to the final concentration of ethanol in the tube, i.e. the higher final concentration of ethanol will produce the higher yield in spheres.

The divided-portion approach in this invention is not obvious and is in fact contrary to the prior art teachings for the following reason. (1) The prior arts (including many of Yen's prior disclosures) have consistently taught that the addition of a desolvating agent (e.g. ethanol solution) above and beyond a critical amount to a protein solution (containing the critical amount of surfactant) will lead to irreversible formation of aggregates which cannot be separated back into individual single spheres (such as the condition in tube 3 here.) The divided-portion approach here has clearly demonstrated that the prior art teaching is no longer true given the discovery in this invention (e.g. in tube 17 here). The volume of ethanol (70%) added to tube 17 is 600 microliters, which is 171% that of the volume (350 microliters) added to tube 3 which formed aggregates. (2) In conventional definitions: a divided portion means dividing an effective volume of the desolvating agent (e.g. 325 microliters, such as in tube 2) into smaller fractions, the sum of which will be the same as the un-divided portion (which is 325 microliter.) However, that is not the teaching here. The teaching of this invention is (a) the first portion is not any random portion, but an amount which is sub-effective. “Sub-effective” means not having a concentration that is capable of producing turbidity in the reaction mixture. In this case, when 250 microliters were added (in tube 11 to tube 17) no spheres were formed: the tubes were clear and not even mildly turbid. The sum of the first portion and the second portion will exceed significantly from the single-portion which has been shown to be effective, above which aggregates will form. However, with the divided-portion approach, even with the supra-volume or supra-mass (defined as a volume or mass that would be damaging in the one-step approach) the total added can be 171% that of an otherwise damaging volume (or mass) if added in one single step. (3) There must be a time interval between the addition of the first portion and the second portion so that the protein molecules in solution can be suitably prepared by the presence of a sub-effective concentration (i.e. a concentration that will not lead to precipitation of soluble molecules into solids) of the desolvating agent, so that when the second portion of desolvating agent is added, useful products (and not aggregates) can be formed and formed with high yield. The time interval between the first and the second portion addition in this experiment was 90 seconds but it can potentially be shorter (e.g. 15 seconds) or much longer (e.g. one hour). (4) Although the approach is called divided-portion, it is not a simple matter of dividing the desolvating agents into portions. For example, tube 5 had been added 400 micoliters of ethanol solution; so had tube 13. But the results were completely different: the contents of tube 5 could obstruct blood vessels if injected into the blood stream of a patient, leading to chest pain, a stroke or even death; whereas the contents of tube 13 have medicinal benefits with few side effects. Therefore, the mathematical similarity (the sum of ethanol being equal in those two tubes) between the two methods does not produce similar results—an obvious case of non-obviousness.

It is impressive that spheres can be formed in the absence of added detergents or surfactants to the protein solution, as taught in the prior art, yet no aggregates were formed if the desolvation agent (ethanol solution) was added in divided steps. It should be noted that in the divided-step method, the first portion of the desolvation agent must be a non-precipitation amount, i.e. to result in a concentration in the mixture unable to form protein precipitates by itself (but can produce spheres when the second portion is added.) Typically this first portion is about 85% (or a lesser quantity) of the desolvating agent that would lead to mild turbidity when added in one-step to the protein suspension, e.g. when 300 microliter of an alcohol solution will produce mild turbidity when added in a single-step to the protein solution, a good start with the divided-portion approach is to use about 250 microliters as the first-portion, to be followed by various other volumes as the second-portion to form the useful spheres.

It should be noted that in many prior art disclosures, a mixture of sizes of spheres are formed at the time of synthesis. Although efforts can be made after synthesis of the spheres to narrow the size range of the particles, such as by filtration or centrifugation to remove the unwanted fractions, significant loss of yield and significant introduction of contaminants can occur with these additional steps. The present invention results in the formation of spheres in a suspension all of which are less in one micron in diameter; the size distribution is a normal distribution with only one peak, which does not require additional steps to remove the unwanted peaks.

### Experiment Two

### The Optimal Time of Adding Fibrinogen to the Albumin Sphere Suspension

Purpose: To find out the optimal amount of time after the formation of spheres to allow spheres to stabilize and not redissolve upon the addition of a fibrinogen solution.

Materials and Methods: Preliminary data had indicated the use of a sub-effective concentration (i.e. a concentration of the desolvating agent too low to prevent the resolubilization of the spheres when the alcohol concentration is reduced) of a glutaraldehyde solution (GL) added to a protein solution has the effect of producing very uniform-sized spheres. However, to stabilize the spheres against resolubilization when the desolvating agent is reduce (or removed) by dilution with fluids not containing the desolvating agent, a second portion of the linking agent (e.g. glutaraldehyde) must be added later to the sphere suspension or be present in the desolvating agent (pre-mixed into the desovlating agent.)

The method used in tube 17 of Experiment One was modified as follows: (1) 1 ml of HSA (7%) was mixed with 1 ml of GL (the sub-effective concentration being 0.125 mg/ml in water) at time zero. (2) At time 60 seconds (after addition of GL to the protein solution) the first portion of desolvating agent (ethanol 70% containing an effective concentration which is 0.5 mg of GL per ml) was added and mixed well with the protein-GL solution. The volume of the first portion was 2.5 ml, which did not produce any turbidity in the tube. (3) At time 250 second (after addition of GL to the protein solution) the second portion of desolvating agent (ethanol 70% containing also 0.5 mg of GL per ml) was added, the volume being 3.5 ml. Thereafter the turbid suspension was divided into aliquots (200 microliters each tube) and 800 microliters of water was added at various time to evaluate if the spheres had become completely stabilized against redissolving (in a solution where the effective ethanol concentration was diluted by a factor of 5 with water.)

Results: Addition of water to a sphere suspension within 10 minutes of the formation of spheres (i.e. appearance of turbidity in the suspension) will visibly decrease the turbidity of the suspension. Assay of the concentration of spheres revealed that the yield in these tubes were only less than 5% to 10%. This confirms that the spheres formed in an adequate concentration of desolvating agents needs at least 10 minutes to stabilize. The yield of the spheres in the suspension (resistance to redissolving) increased with time between 15 minutes and reached complete stability in one hour (reaching a plateau of maximal yield of about 99%.)

The experiment was repeated with the addition of a fibrinogen solution (between 1 mg and 2 mg fibrinogen/ml) to the sphere suspension. It was found that for one volume of albumin sphere suspension, the optimal volume of fibrinogen solution to be added is between one-fifth volume to one-third volume. Also, the optimal time to add the fibrinogen solution is about one hour after the start of the experiment (i.e. the adding of a GL solution, at 0.125 mg GL/ml to the protein solution.) The size of the spheres did not appear to change after the addition of the volume of fibrinogen solution to the volume of sphere suspension; they remain less than one micron. No aggregate were observed by microscopic examination. The sphere suspension thus prepared was stable for at least 3 days when stored in refrigerated temperature without the formation of aggregates. In contrast, the suspension formed by the single-step method (with or without the addition of fibrinogen) tended to form aggregates upon prolonged storage, unless the ethanol was removed within 6 hours of the formation of the spheres.

Administration of the suspensions of fibrinogen-coated spheres to thrombocytopenic animals (with less than 1% of the platelet concentration of their healthy counterparts) is highly efficacious. The data showed that at a dose of 4 mg spheres/kg or higher, administered intravenously, the suspensions are effective in reducing the bleeding time and the bleeding volume of these animals. It is expected that prophylactic administration of the suspension to patients who are not yet thrombocytopenic but are expected to suffer large blood loss (such as patients about to have a difficult surgical operation, or trauma patients in active bleeding who are not yet thrombocytopenic but soon will become thrombocytopenic) will also reduce the amount of blood loss in these patients during surgery or during the episode of blood-loss, and afterward.

Comments: The yield of any industrial process must be optimized. It is found here that the best time to add the fibrinogen solution is about one hour after the addition of the GL solution to the protein solution. Adding the fibrinogen solution sooner than one hour can lead to a lower yield of the spheres due to the redissolving of the spheres which have not been completely stabilized. The volume of the fibrinogen solution should be as small compared to the volume of the sphere suspension so as not to over-dilute the concentration of the spheres: the volume of the fibrinogen solution being one third that of the volume of sphere suspension is ideal.

This requirement of about 60 minutes in between the two points in the manufacturing process (addition of fibrinogen to be delay by about one hour after appearance of turbidity) will make it extremely difficult to use a tubing system for mass production (as disclosed in the prior art by Yen.) A very long tube has to be installed to allow the portion of partially cross-linked spheres to move to the next point in the tubing system (the Y-junction) where fibrinogen molecules can then combine with the now-stabilized spheres. A very long tube is expensive and will lead to a large amount of waste (partially processed material in the “dead space”) within the interior of the tubing. If the tube is long, friction with the wall of the tube will cause the material closest to the wall to move slower than the material at the center of the tube; thus the time taken to move material from one location to the next location can be very different from what is expected from the “pump rate.” If the tube is particularly long, there will be areas where the tube will be curved or bent, causing very uneven flow of material around the corner—thus defeating the primary purpose of the tubing system which is to allow addition of new material at fixed time points in the manufacturing process. All these difficulties are overcome by the batch-mixing method of the present invention to be disclosed in the following experiment.

### Experiment Three

### Mass Production of a Ready-to-Use Formulation of Fibrinogen-Coated Albumin Spheres in Quantities of at Least 100 Liters

Purpose: to evaluate the success of the divided-portion approach to sphere formation using large quantities of materials.

Materials and Methods: The method of Experiment Two was scaled up 10,000 times. All the containers used were sterile and had been depyrogenated by heat. The density of the various ingredient solutions was obtained by weighing a known volume of the solution. The experiment described here quotes volume measurements. However, during the actual performance of the manufacturing process, the exact volumes were dispensed or mixed by their weight, which was more accurate to measure than volume measurements. The weight of material that was to be pumped into a container was obtained by convertion from the known density of the material. Essentially: (1) 10 liters of HSA (7%) was pumped into a stainless steel drum (50 gallon capacity). (2) At time zero, 10 liters of GL (0.125 mg/ml) was added to the drum and well shaken with a specially designed platform-shaker capable of agitating quickly the contents of the 50-gallon drum. (3) At time equals to one minute, 25 liters of the first portion of desolvating agent (70% ethanol containing 0.5 mg GL/mg) was added. (4) at time equals to 2.5 minutes, 35 liters of the second portion of desolvating agent was added (same composition as the first portion). The suspension turned turbid. (5) At time equal to one hour, 20 liters of a fibrinogen solution (1 mg per ml) was added to the turbid solution. (6) The fibrinogen-coated albumin sphere suspension was then stored overnight at 5 to 9 degrees Centigrade in sterile condition.

Thereafter the suspension was dialyzed to remove as much alcohol as possible. A sterile sorbitol solution was added to achieve a 5% sorbitol in the final suspension (to maintain osmolarity compatible with blood.) A sodium caprylate solution (1%) was added to achieve a final concentration of 13.3 mg caprylate per mg protein (sphere plus soluble proteins) in the suspension. Sodium caprylate is known to provide protection of soluble protein molecules against heat denaturation. The concentration of the spheres was adjusted to 8 mg spheres/ml of the suspension.

Aliquots of 100 ml each were dispensed into each 100-ml glass vial and capped. Terminal sterilization was pertained at 65 degree Centigrade for 12 hours. Long term storage was carried out in minus 20 degrees, in 5 to 9 degrees, in 20 to 25 degrees and in 40 to 42 degrees Centigrade.

Results:

Examination under the microscope and analysis of particle size by laser technology showed that all the spheres formed by the two-step method were less than one micron in diameter and had a normal distribution with only one peak. This is in sharp contrast to the result of having several peaks of spheres (including a population larger than 7 micron in diameter) from using the method disclosed by Yen in U.S. Pat. No. 6,264,988 “Fibrinogen-coated Microspheres”.

Although particular concentrations of HSA, GL and alcohol concentrations are quoted here, it is obvious that a range of concentrations of these agents (plus or minus at least 20% of the quoted values) can be used effectively. The room temperature during this experiment was 20 to 21 degrees Centigrade; but a lower or higher room temperature can be tolerated.

Sodium caprylate has been used to protect soluble proteins from denaturation by heat. It is not obvious that this compound can protect proteins in a solid form, such as a protein sphere bonded in such a way that the individual molecules at various locations in the sphere might or might not have become more susceptible to heat denaturation. Therefore, the usefulness of caprylate in this case is not obvious from the prior art.

Pyrogen content and sterility of the vials were studied after one year of storage in room temperature. The vials were shown to be free of pyrogens and infectious agents even after this duration of storage.

Administration of one ml of the suspension (i.e. containing 8 mg spheres per ml) per kilogram weight of animals that are severely thrombocytopenic showed that the suspension is effective in reducing bleeding time and the volume of blood loss, in reducing the amount of ecchymosis and in reversing the formation of petechiae in these animals. These thrombocytopenic animals typically have less than 1% of the normal concentration of endogenous platelets and have a great tendency toward spontaneous internal bleeding.

For other indications, such as treatment of burn patients, septic patients, DIC patients or patients exposed to platelet-depleting viruses, or after a lethal dose of irradiation, the effective dose of fibrinogen-coated albumin spheres may be different. Depending on the indication, the dose may be as high as 32 mg per kg weight of the patient or may be as low as 2 mg per kg weight of the patient.

There is no evidence of the formation of neoantigens on the spheres from the heat inactivation.

Careful measurement of the size of spheres in the mass-produced suspensions revealed that less than 0.1% of the spheres may be slightly larger than one micron in diameter. While these slightly larger spheres are not expected to obstruct any capillaries (which are about 6 to 7 micron in diameter) they may settle theoretically to the bottom upon prolonged storage in a container. However, it was found that if the concentration of spheres in the container is 4 mg spheres/ml or higher, the high concentration of spheres (which is about several trillions of particles per ml) which are less than one micron in diameter will keep the slightly-larger-than-one-micron spheres in suspension without the formation of any bottom layers after storage of the bottles for at least 6 months. Storage in refrigeration conditions is expected to result in substantially longer shelf-life since the refrigerator tends to vibrate during cycles of cooling.

Comments: The success of this mass-production scheme involves more than the fact that spheres can be produced in mass quantities, or that the spheres have size distributions or other qualities similar to batches produced in more well-controlled smaller quantities. The handling of gallons of material, the need to assure absolute sterility and non-pyrogenicity of the equipment and solutions, the tendency of the material to splash and spill, all pose challenges. Therefore, it is included in this invention a method of terminal sterilization that will ensure that even if some known or unknown infectious agents have inadvertently entered into the suspension, the infectious agents can be inactivated without harm to the spheres and the excipient components, such as by using the method to be described below.

### Experiment Four

### The Effect of Heat Treatment on the Inactivating of Infectious Agents Added to the Protein Solution Before the Addition of the Desolvation Agent

Purpose: To confirm that even if infectious agents were hiding in the interior of the spheres, the heat inactivation step in the terminal sterilization procedure can inactivate the infectious agents without damage to the spheres and the excipient components.

Materials and Methods: Suspensions of fibrinogen-coated albumin spheres were prepared according to the method used in Experiment One except that infectious agents were added to the albumin solution before the addition of the desolvating agent to form spheres. The infectious agents used here include enveloped viruses (e.g. DNA viruses such as Herpes viruses, RNA viruses such as Hepatitis-D virus, Retroviruses such as Hepadnaviruses) and non-enveloped viruses (e.g. norovirus, rotavirus and human pappillomavirus—HPV). After the various sphere suspensions were prepared, they were filled into the respective glass bottled which were then capped and sealed. All the suspensions also contain a final concentration of 5% sorbitol and 13.3 mg of sodium caprylate per gram of total protein (soluble protein plus spheres) to protect the protein molecules against heat denaturation. The bottles were placed in a hot water bath and heat-treated at 65 degrees Centigrade for 12 hours. Thereafter aliquots of the sphere suspensions were aseptically removed from the glass vial; the spheres were dissolved by treatment with a sterile protease solution to release any infectious particles potentially trapped within the spheres. Titers of infectious agents were assayed and compared to positive and negative controls.

Results: The data showed that positive controls were positive and negative controls were negative and the spheres seeded with infectious agents as described above were completely non-infectious. It showed that the heat-inactivation was effective in the inactivation of infectious agents including enveloped viruses and non-enveloped viruses. The heat treatment did not change the size of the spheres in the suspension, nor did administration of the heat-treated spheres intravenously into test animals result in any adverse clinical signs and symptoms. Administration of heat-treated suspensions to thrombocytopenic animals showed benefits similar to the administration of control, non-heat-treated suspensions which had not been seeded with infectious agents.

Comments: Yen had disclosed inactivation of viruses and other infectious agents by extreme high pressure (see US Patent Application Publication Number 2011/0251127 A1, published on Oct. 13, 2011.) Yen disclosed that inactivation by pressure will not work in glass containers which will break under pressure. However, most commercial glass “serum” bottles can withstand heat without cracking. The data from this experiment showed that viruses that potentially can hide inside a sphere can be inactivated by the heating conditions described above even when applied to the suspension sealed inside a glass container.

It is believed that other infectious agents such as fungus, bacteria, viroids, satellites, prions, as well as derivatives of some of these agents—the spores and mutated proteins of these agents that potentially can be introduced during the manufacturing process of the ready-to-use suspension can also be inactivated by the heat treatment in this invention without resulting in any damage to the medical benefit of the suspension and without introduction of side-effects or adverse reactions to the patient.

Sorbitol used as an excipient here is particularly useful for heat treatment. Other commonly used excipient compounds, such as glucose, maltose, or lactose, will turn dark brown (caramelization) after prolonged heat treatment, rendering the suspension dirty-looking which will be rejected by health professionals. Such degradation of the excipient component by heat, whether in chemical structure, or even just in coloration, will not be acceptable in a medical product. The suspension containing sorbitol in this invention looks mildly yellow after the heat treatment. It is harmless to the patient and more importantly does not offer protection to the infectious agents against the heat inactivation treatment. Although sorbitol is used in the present experiment, it is believed that other excipient components which do not become discolored or become denatured by heat and which do not offer protection to infectious agents can be used.

This experiment here used spheres coated with fibrinogen. It is expected that other kinds of spheres can also be subjected to heat treatment to inactivate infectious agents successfully without damage to the medical effectiveness of the spheres, including spheres that carry other biological molecules, drugs, chemicals, DNA, RNA and radiolabeled tracing material, or even blank spheres not having any other molecules added to them during the manufacturing process.

### Experiment Five

### The Effect of Heat Treatment on the Inactivation of Infectious Agnets Added to the Sphere Suspension After the Spheres have been Formed

Purpose: to evaluate the success of heat treatment in the inactivation of infectious agents added to the sphere suspension after the formation of the spheres during the manufacturing process.

Materials and Methods: The same materials and methods were used as in Experiment Four except that the respective infectious agents were added not to the protein solution but to the turbid suspensions after the spheres were formed—so that a high titer of infectious agents has the opportunity to attach to the surface of the spheres, with potential protection against heat-inactivation of the proteins and the genomic material of the infectious agents, by virtue of their close proximity to the surface of a protein sphere.

Results: the results were similar to that of Experiment Four. It should be noted that “success” in heat inactivation includes not just the fact that the infectious agent becomes inactivated but that the spheres and the incipient components of the suspension are not adversely affected to any way by the heat treatment.

Comment: the complete success of heat-treatment is particularly important to a ready-to-use suspension of protein spheres. If the heat-treatment is only partially successful, any infectious agent, such as a single bacterium that has survived the treatment, can have the chance to grow in this rich medium for a long time, up to a year. The turbidity of the suspension, due to the presence of the spheres will obscure the fact that some of the “particles” in such a suspension can be live bacteria. When administered to the patient, the presence of viruses, or bacteria and their toxins will cause great harm to the patient, which must not be allowed to happen and which has been shown to be preventable by the heat-treatment of this invention.

### FURTHER SUMMARY AND COMMENTS ON THE INVENTION

The invention comprises both a composition invention and a method invention. The composition is a suspension, not just the spheres in the suspension, but both the spheres and the supernatant of the suspension. Further comments and explanations are provided below.

It is a composition comprising a suspension which further comprises of (a) fibrinogen-coated albumin spheres and (b) a supernatant, where the spheres do not sediment to form a layer within six month in the supernatant, the spheres are always in contact with an aqueous phase medium since the synthesis of the spheres, said spheres have not been exposed directly to air, and said suspension is effective in the treatment of patients with bleeding problems related to platelets.

This invention is distinguished from the prior art in at least the fact that a single population of spheres is formed which has a normal distribution ranging from about one micron to less than 0.1 micron—it has only one peak. In contrast, the prior art composition often had more than one peak at the time when the spheres were first synthesized, necessitating further steps of “purification” such as by filtration or centrifugation to remove spheres of the unwanted sizes.

The fact that spheres of the present invention do not settle to the bottom during prolonged storage indicates that the density of the spheres (weight of a sphere divided by the volume of a sphere) is very close to a value of one gram per cubic centimeter of volume, which is the density of water or of most aqueous solutions. The spheres are suspended in the supernatant by the Brownian movement of the molecules in the supernatant. The spheres do not float to the top of the container during long term storage, indicating that their density is not less than 1.00. It is expected that the density of the spheres within the population is 1.00 to 1.10 and the density of the supernatant fraction is also between 1.00 and 1.10 gram per cubic centimeter.

Whether any of the spheres have formed a sediment can be evaluated by visual inspection of the bottom of the glass container after a period of storage; or by measurement of the turbidity (or concentration of spheres) of the top fraction of the stored suspension, said top fraction will have decreased turbidity and concentration of spheres compared to the bottom fraction of the stored suspension.

Although the spheres are always suspended in an aqueous suspension since their synthesis, the aqueous medium in which they are synthesized contains a high concentration of alcohol which may not be suitable for use in some patients. The synthesis medium is also not adjusted for compatibility in osmolarity with blood. Excessive amounts of alcohol need to be removed, after which the appropriate excipient components need to be added back to the suspension to render the suspension compatible with heat treatment and intravenous administration. The “supernatant” in this invention refers not to the medium in which the spheres are synthesized, but to the aqueous medium in the final suspension to be filled into a glass container to undergo terminal sterilization; and to the aqueous medium in which the spheres are suspended, which has undergone the terminal sterilization without damage to either the spheres or the supernatant (the heat-treated spheres and the heat-treated supernatant are collectively to be called the final product.)

It is also a composition comprising a suspension of fibrinogen-coated albumin spheres and a supernatant where the spheres at the time of synthesis are all less than one micron in diameter, said population of spheres do not sediment to form a layer within twelve months in said supernatant, said spheres are always in contact with an aqueous phase medium and have not been exposed directly to air, and said suspension is effective in the treatment of patients related to platelets.

It is also a composition where said aqueous phase medium comprises an excipient component which renders the suspension compatible with blood in osmolarity and which is not degraded by heat treatment. This invention involves not just the spheres, but also the components in the supernatant, i.e. that of the excipient component. Both spheres and the supernatant are important to the success of the invention because the product comprises of both the spheres and the excipient molecules in the suspension, both of which have to undergo a heat treatment for terminal sterilization.

This is a composition where said suspension including the excipient components is further subject to heat treatment under a condition where infectious agents are inactivated but where the spheres and the excipient components in the suspension are not damaged and will not cause antibody formation in a human body.

This is a composition where the heat treatment comprises heating the suspension inside a glass container to at least 60 degree centigrade, possibly to 65 degree centigrade, and for at least 10 hours, possibly 12 hours.

This is a composition where the suspension is effective in the treatment of patients who are numerically thrombocytopenic. The standard definition of thrombocytopenia is “a condition in a patient who has a platelet concentration that is numerically below normal concentrations” which means in the human patient less than 150,000 platelets per microliters of blood. We use the term “numerical thrombocytopenia” in this invention to distinguish what is called “functional thrombocytopenia,” as explained below.

This is also a composition where the suspension is effective in the treatment of patients who are functionally thrombocytopenic. One class of patients with bleeding problems related to platelets is the cardiovascular patients who are administered anti-platelet medications to prevent chest pains, heart attacks or strokes. Compounds such as aspirin and clopidogrel will irreversibly inhibit the function of platelets upon contact with the drug (or its short-lived derivatives) but the drug will not cause the removal of the inactivated platelets from the body. Therefore, numerically these patients will show no decrease in the total concentration of platelets in the blood. Fortunately, the body continues to produce fresh platelets constantly which have not been inactivated. Because the patient takes the medication only once a day and not continuously throughout the day, there will always be some functional platelets in the body and the patient will not bleed excessively. The longer away in time from the last dose of anti-platelet medication, the more functional-platelets will be in the body. A numerical measurement of the concentration of platelets in these patients will not reveal the percent of platelets that are non-functional (or functional). We use the term “functional” thrombocytopenia to describe these patients.

This is also a composition where the suspension is effective in the prophylactic treatment of patients who are expected to become thrombocytopenic or can benefit from a higher concentration of particles that can plug the wounds in blood vessels. Such patients include surgical patients who will undergo difficult surgeries; or volunteers who will go into a radioactive and contaminated area to do repair or to rescue victims. Exposure to lethal doses of radiation will destroy the capacity of the bone marrow to produce platelets and other blood cells and the exposed person will become thrombocytopenic in about one week.

This invention is also a method of mass production of suspensions of albumin spheres where the spheres have a population size-distribution at the time of synthesis of the spheres ranging from about one micron to less than 0.1 micron in diameter, with less than 0.1% of said spheres having a size greater than one micron, said spheres are always in contact with an aqueous phase medium and have not been exposed directly to air, comprising,

dissolving albumin molecules in an aqueous solution without the presence of a surfactant or detergent;

addition of a crosslinking solution which results in a sub-effective concentration for the complete crosslinking of spheres by the crosslinking agent;

(subeffective is defined here as “not capable of or not effective in” preventing the re-dissolving of the spheres when the concentration of the desolvating agent is decreased in the subsequent steps, such as from dilution by water, or from dilution by the addition of a fibrinogen-containing solution which does not have the desolvating agent in the fibrinogen-containing solution.);

addition of a first portion of desolvating solution which results in a concentration of the desolvating agent insufficient to cause persistent turbidity of the mixture;

(in other words, if persistent turbidity appears after the addition of this first portion, the desolvating solution is excessive and has already produced irreversible spheres. Non-persistent turbidity is permissible because when the first portion of the desolvating agent is in the process of being added to the mixture of protein-and-crosslinking-solution, there will be localized and uneven distribution of the various components, i.e. some local areas will have high concentrations of desolvating agent temporarily reacting with the protein molecules there. Such temporary turbidity caused by inadequate mixing will immediately redissolve upon further shaking of the container to evenly distribute the components. Typically, temporary turbidity will disappear within 15 seconds of the shaking of the container to mix well the ingredient solutions.); and

addition of a second portion of desolvating solution after a waiting period which results in a combined concentration of the desolvating agent sufficient to cause the formation of spheres stable against redissolving and without the formation of aggregates.

This second portion will cause the formation of spheres. Although the crosslinking agent in the (b)-step is not sufficient by itself to cause complete stabilization of the spheres against redissolving, the presence of a large amount of desolvating agent in this case (the combined concentration of the first portion with the second portion) will provide some stability to the spheres thus formed, to be stable against immediately re-dissolving. It should be noted that the combined concentration of the first and second portion of desolvating agent in this invention is far more than can be tolerated by the amount of desolvating agent used in the one-step prior art method. The vast amount of the desolvating agent in the present invention has some stabilizing effect by itself on the spheres against resolubiliztion.

Even so, to be sure that the spheres do not redissolve after one hour, additional amount of crosslinking agent can be added to both the first portion of the desolvating agent and the second portion of the desolvating agent. In the experiments described, the desolvating agent also contains 0.5 mg of glutaraldehyde per ml of the desolvating solution.

In this experiment, the recorded time of the addition of an ingredient solution is the time at the beginning of the pouring of that specific ingredient solution into the 50-gallon drum. The amount of time needed to completely pour the first portion of the desolvating agent (25 liters, as in Experiment Three) into the 50 gallon drum is typically more than 30 seconds. Therefore, the time between the beginning of the pouring of the first portion and the beginning of the pouring of the portion must be longer than 30 seconds (depending on the exact amount to be poured.) In other words, there should be a minimal time of waiting, say at least 15 seconds for the complete mixing of all the ingredient solutions that have been added so far into the 50 gallon drum. Experiment Three allowed 90 seconds between the beginning of pouring the first portion of the desovating agent and the beginning of the pouring of the second portion—which allows adequate mixing of all the ingredients in the 50 gallon drum. Having an adequate time in between the addition of the first and the second portion of the desolvating agent is very important. This period of time will allow the first portion of the desolvating agent to adequately prepare the surface of the still dissolved albumin molecules so that they can tolerate the large amount of the second portion of desolvating agent without causing the formation of aggregates—even a smaller amount of desolvating agent would have caused massive amounts of aggregates if the desolvating solution were added in one-step, such as was used in the prior art methods.

The invention also includes a method where said mass production of suspensions of albumin spheres includes an additional step,

Addition of a solution containing fibrinogen to the suspension of albumin spheres after a waiting period of about one hour after the addition of a second portion of desolvating solution, to result in suspensions of fibrinogen-coated albumin spheres.

The invention is a method where the yield of spheres in the suspension exceeds 80%.

The yield is defined as the concentration of spheres divided by the concentration of total proteins (total protein means spheres plus the residual soluble proteins in the supernatant) typically in one ml of suspension.

The invention is a method where the yield of spheres in the suspension exceeds 95%. (The yield of the prior art using the one-step method is typically only less than 30%.)

The invention is a method where said waiting period between the completion of the addition of first portion of desolvating solution and the beginning of addition of the second portion of desovlating solution exceeds 15 seconds.

The invention is a method where the concentration of spheres at the time of synthesis of the spheres exceeds one trillion spheres per ml of the suspension.

(The concentration of spheres in the final product is about 8 mg to 12 mg spheres per ml of suspension. There are very few instruments that can count the number of particles of this small size accurately. However, the weight of a sphere with a diameter of 0.4 micron can be calculated, using an estimated density of one gram per cubic centimeter. The calculation will show that the concentration of spheres with a median diameter of 0.4 micron in one ml of suspension containing 8 mg spheres/ml of suspension will exceed one trillion spheres per ml of the suspension.)

The invention is a method of mass production by pouring ingredient solutions into a large drum, where the volume of said suspension at the time of the formation of the spheres exceeds 50 liters. (Experiment Three described adding 10 liters of GL to 10 liters of albumin solution, then 25 liters of first portion, followed by 35 liters of second portion of desolvating agent, to a total of 80 liters. The method is easily applicable to produce albumin suspensions from 50 liter to 500 liters or even larger volumes.)

The invention is method of mass production of a ready-to-use suspension where the desolvating agent is ethyl alcohol and where the concentration of said ethyl alcohol at the time when the spheres are synthesized is at or above 45% in the suspension.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

What is claimed is:

1. A composition comprising a suspension which is further comprised of fibrinogen-coated albumin spheres and a supernatant, where the spheres do not sediment to form a layer within six months in a supernatant, said spheres have a relative density between 1.00 and 1.10, said spheres are always in contact with an aqueous phase medium since their synthesis from soluble proteins and have not been exposed directly to air, and said suspension is effective in the treatment of patients with bleeding problems related to platelets.

2. A composition according to claim 1, where said supernatant comprises an excipient component which renders said suspension compatible with blood in osmolarity and which is not degraded by heat treatment.

3. A composition according to claim 1, where said spheres and said supernatant are further subject to heat treatment under a condition where infectious agents are inactivated and where said spheres and said supernatant are not damaged and will not cause antibody formation in a human body.

4. A composition according to claim 3, where the heat treatment comprises heating said spheres and said supernatant inside a glass container to between above 60 degrees centigrade and 65 degrees centigrade, for between at least 10 hours and 12 hours.

5. A composition according to claim 1, where said suspension is effective in said treatment of patients who are numerically thrombocytopenic.

6. A composition according to claim 1, where said suspension is effective in the treatment of patients who are functionally thrombocytopenic.

7. A composition according to claim 1, where said suspension is effective in the prophylactic treatment of patients who are expected to become thrombocytopenic.

8. A composition comprising a suspension which is further comprised of fibrinogen-coated albumin spheres and a supernatant, where the spheres at a time of synthesis are all less than one micron in diameter, said spheres do not sediment to form a layer within twelve months in the supernatant, said spheres have a relative density between 1.00 and 1.10, said spheres are always in contact with an aqueous phase medium since their synthesis from soluble proteins and have not been exposed directly to air, said supernatant has a density between 1.00 and 1.10 and said suspension is effective in the treatment of patients related to platelets.

9. A composition according to claim 8, where said supernatant comprises an excipient component which renders said suspension compatible with blood in osmolarity and which is not degraded by heat treatment.

10. A composition according to claim 8, where said spheres and said supernatant are further subject to heat treatment under a condition where infectious agents are inactivated and where said spheres and said supernatant are not damaged and will not cause antibody formation in a human body.

11. A composition according to claim 10, where the heat treatment comprises heating said spheres and said supernatant inside a glass container to between above 60 degrees centigrade and 65 degrees centigrade, for between at least 10 hours and 12 hours.

12. A composition according to claim 8, where said suspension is effective in the treatment of patients who are numerically thrombocytopenic.

13. A composition according to claim 8, where said suspension is effective in the treatment of patients who are functionally thrombocytopenic.

14. A composition according to claim 8, where said suspension is effective in the prophylactic treatment of patients who are expected to become thrombocytopenic.

15. A method of mass production of suspensions of albumin spheres by mixing ingredient solutions in batches, where the spheres are always in contact with an aqueous phase medium since their formation and have not been exposed directly to air, where said spheres remain completely in suspension and do not sediment after storage for at least six months, comprising:
a. dissolving albumin molecules in an aqueous solution without the presence of a surfactant or detergent;
b. adding a crosslinking solution which results in a concentration of the crosslinking agent that is insufficient for complete crosslinking of spheres by the crosslinking agent;
c. adding a first portion of desolvating solution which results in a concentration of the desolvating agent insufficient to cause persistent turbidity of the mixture; and
d. adding a second portion of desolvating solution after a waiting period which results in a combined concentration of the desolvating agent sufficient to cause the formation of spheres stable against redissolving and without the formation of aggregates.

16. The method according to claim 15, where said mass production of suspensions of albumin spheres includes an additional step of:
a. adding a solution containing fibrinogen to the suspension of albumin spheres after a waiting period of about one hour after the addition of a second portion of desolvating solution, to result in suspensions of fibrinogen-coated albumin spheres.

17. The method according to claim 15 or 16, where the yield of spheres in said suspension exceeds 80%.

18. The method according to claim 15 or 16, wherein the yield of spheres in said suspension exceeds 95%.

19. The method according to claim 15 or 16, where said waiting period between the completion of the addition of first portion of desolvating solution and the beginning of the addition of the second portion of desovlating solution exceeds 15 seconds.

20. The method according to claim 15 or 16, where the concentration of spheres at the time of synthesis of the spheres exceeds one trillion spheres per ml of the suspension.

21. The method according to claim 15 or 16, where the volume of said suspension at the time of the formation of the spheres exceeds 50 liters.

22. The method according to claim 15 or 16, where the desolvating agent is ethyl alcohol and where the concentration of said ethyl alcohol at the time when the spheres are synthesized is at or above 45% in the suspension.
